(19) United States
(12) Patent Application Publication
(10) Pub. No.: US 2022/0375603 A1
(43) Pub. Date: Nov. 24, 2022

(54) SYSTEMS AND METHODS FOR IMPLICIT DISABILITY DIAGNOSIS

(71) Applicant: BIZZ dot BUZZ, LLC, Beaumont, TX (US)

(72) Inventor: Shane Taylor McFarland, Beaumont, TX (US)

(21) Appl. No.: 17/566,887

(22) Filed: Dec. 31, 2021

Related U.S. Application Data
(60) Provisional application No. 63/133,212, filed on Dec. 31, 2020.

(51) Int. Cl. G16H 50/20; G16H 10/20

(52) U.S. Cl. CPC G16H 50/20; G16H 10/20

(57) ABSTRACT

The systems and methods disclosed herein are for a implicitly diagnosing disability. The systems and methods comprising receiving a first signal, receiving a responsive first input, executing a first program for a predetermined amount of time, displaying a first question, determine if a second response is received, displaying a second question and receiving a second response, and computing a disability diagnosis based on the received input.

### CROSS-REFERENCE TO RELATED APPLICATIONS

This application claims priority to and benefit of U.S. Provisional Patent Application No. 63/133,212, titled “SYSTEMS AND METHODS FOR IMPLICIT DISABILITY DIAGNOSIS,” filed on Dec. 31, 2020. That application is incorporated by reference in its entirety herein.

### BACKGROUND

### Field of the Art

This disclosure relates generally to testing and more specifically to automatically monitoring for disabilities while performing other testing.

### Discussion of the State of the Art

Increasingly, children are using electronic devices to learn. The children may access lessons and/or quizzes that test understanding of a subject. When a child answers a question incorrectly, it may be because the child does not understand the subject. However, the child may have incorrectly answered the question for another reason, such as a disability that prevents the child from answering the question correctly. What is needed is a system and/or method that can monitor for disabilities while testing children's knowledge of subjects.

### SUMMARY

The present invention utilizes systems and/or methods for implicit disability diagnosis. In an example method, a signal indicative of instructions to execute a program may be received. In the example method, a first question may be retrieved. In the example method, the program may execute for a first predetermined amount of time. In the example method, after the first predetermined amount of time, execution of the program may be paused and the first question may be displayed. In the example method, a response to the first question may be received. In the example method, a response to the first question may be received until a correct response is received. In the example method, once a correct response is received, a second question may be retrieved based at least in part on the received one or more response(s) to the first question. In the example method, the program may execute for a second predetermined amount of time. In the example method, after the second predetermined amount of time, execution of the program may be paused and the second question may be displayed. In the example method, a response to the second question may be received. In the example method, a likelihood of a condition may be determined based on the responses to the first question and the second question.

One benefit of the present invention is that monitoring of disabilities may happen simultaneously and automatically while testing a student's knowledge of a subject. This is a faster and more accurate way to test a child for a condition that could impair an ability to perform a particular test. Additionally, this reduces the computational resources needed for disability diagnosis as a need for separate testing is eliminated or reduced.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a system for implicit disability diagnosis in accordance with an exemplary embodiment of the invention.

FIG. 2 illustrates an implementation of a user device in accordance with an embodiment of the invention.

FIGS. 3A and 3B illustrate a flowchart for implicit disability diagnosis in accordance with an exemplary embodiment of the present invention.

FIG. 4 illustrates an exemplary computing device that supports an embodiment of the inventive disclosure.

FIG. 5 illustrates an exemplary standalone computing system that supports an embodiment of the inventive disclosure.

FIG. 6 illustrates on embodiment of the computing architecture that supports an embodiment of the inventive disclosure.

FIG. 7 illustrates an exemplary overview of a computer system that supports an embodiment of the inventive disclosure.

### DETAILED DESCRIPTION

The inventive systems and methods (hereinafter sometimes referred to more simply as “system” or “method”) described herein significantly reduce computation resources used to monitor a student for disabilities (e.g., conditions, statuses, etc.). A student may use a device to executing a testing application for testing knowledge on one or more particular subjects. The device may comprise or be associated with additional functionality, such as third-party applications. The student may cause the testing application to activate one of the third-party applications. The testing application may execute the third-party application for a predetermined amount of time. After the predetermined amount of time, the testing application may ask the student a first question. If the student answers the first question incorrectly, the testing application may select a second question, wherein the second question may be used to determine why the student answered the first question incorrectly. For example, if the first question asks a student to identify a green square and the student fails, the second question may ask the student to identify a black square. If the student fails to on the second question, then the testing application may make an inference that the student failed to answer the first question correctly because the student does not know what a square is. If the student correctly answers the second question, then the testing application may make an inference that the student failed to answer the first question correctly because the student is color blind and is not able to see the green square. Once an inference is made about a condition, the testing application may select specific questions to test the inference and strengthen or weaken confidence in the inference.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

### Conceptual Architecture

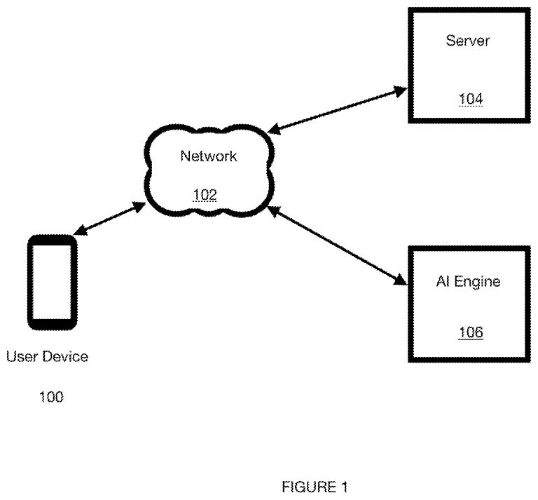

FIG. 1 illustrates a system for implicit disability diagnosis with an exemplary embodiment of the invention. The system may comprise a user device 100, a network 102, a server 104, and an artificial intelligence (AI) engine 106. The various computing devices described herein are exemplary and for illustration purposes only. The system may be reorganized or consolidated, as understood by a person of ordinary skill in the art, to perform the same tasks on one or more other servers or computing devices without departing from the scope of the invention.

The user device 100 (herein referred to as user input device, user device, or client device) may include, generally, a computer or computing device including functionality for communicating (e.g., remotely) over the network 102. Data, such as lessons, quizzes, tests, answers, etc., may be collected from one or more remote servers, such as the server 104 via the network 102. Client device(s) 100 may be a server, a desktop computer, a laptop computer, personal digital assistant (PDA), an in- or out-of-car navigation system, a smart phone or other cellular or mobile phone, or mobile gaming device, among other suitable computing devices. Client devices 100 may execute one or more client applications, such as a web browser (e.g., Microsoft Windows Internet Explorer, Mozilla Firefox, Apple Safari, Google Chrome, and Opera, etc.), or a dedicated application to submit user data, or to make prediction queries over the network 102.

In particular embodiments, each user device 100 may be an electronic device including hardware, software, or embedded logic components or a combination of two or more such components and capable of carrying out the appropriate functions implemented or supported by the user device 100. For example and without limitation, user device 100 may be a desktop computer system, a notebook computer system, a netbook computer system, a handheld electronic device, or a mobile telephone. The present disclosure contemplates any user device as the user device 100. The user device 100 may enable a network user at the user device 100 to access network 102. The user device 100 may enable its user to communicate with other users at other client devices.

The user device 100 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. The user device 100 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 100 one or more Hyper Text Markup Language (HTML) files responsive to the HTTP request. The user device 100 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 100 may also include an application that is loaded onto the user device 100. The application obtains data from the network 102 and displays it to the user within the application interface.

This disclosure contemplates any suitable number of client devices 100, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

The server 104 may comprise question and correct answer pairs. The server 104 may comprise question and answer options sets. The server 104 may receive a request from the user device 100 via the network 102 and may select a question and answer options set in response to the request and transmit the question and answer options set to the user device 100 via the network 102.

The AI engine 106 may be in communication with the server 104 and/or the user device 100 via the network 102. The AI engine 106 may be trained. Users with a known condition (e.g., disability, status, etc.) may be presented with a set of questions. The responses to the set of questions from the users with the known condition may be used to train the AI engine 106 to recognize responses from future users with the known condition. As a user of the user device 100 uses the system, responses to questions may be used to identify or eliminate conditions associated with the user. When responses for a user match a profile of users with a certain condition with a threshold accuracy, the user may be considered to have the certain condition. A certainty that a user has the certain condition may depend on responses and/or response times to questions. Once a user is associated with a condition, future responses to future questions from the user may be used to further train the AI engine 106 regarding the condition and future questions.

The network 102 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 102 is an intranet, an extranet, a virtual private network (VPN), a local area network (LAN), a wireless LAN (WLAN), a wide area network (WAN), a metropolitan area network (MAN), a portion of the Internet, or another network 102 or a combination of two or more such networks 102. One or more links connect the systems and databases described herein to the network 102. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 102, and any suitable link for connecting the various systems and databases described herein.

The network 102 connects the various systems and computing devices described or referenced herein. In particular embodiments, network 102 is an intranet, an extranet, a virtual private network (VPN), a local area network (LAN), a wireless LAN (WLAN), a wide area network (WAN), a metropolitan area network (MAN), a portion of the Internet, or another network or a combination of two or more such networks 102. The present disclosure contemplates any suitable network 102.

One or more links couple one or more systems, engines or devices to the network 102. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 102.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiment, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

### Software Suite for Implicit Disability Diagnosis

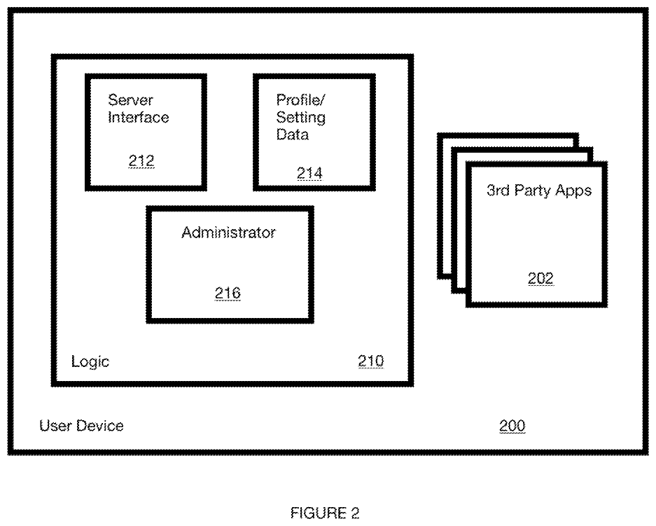

FIG. 2 illustrates an implementation of a user device 200 in accordance with an embodiment of the invention. The user device 200 may be or comprise the user device 100 in FIG. 1. The user device 200 may comprise one or more third-party application(s) 202 and logic 210. The logic 210 may comprise a server interface 212, profile and/or setting data 214, and an administrator 216. Other systems and databases may be used, as would be readily understood by a person of ordinary skill in the art, without departing from the scope of the invention.

The one or more third-party application(s) 202 may comprise any program executable on the user device 200. The one or more third-party application(s) 202 may comprise audio and/or video content. The one or more third-party application(s) 202 may comprise one or more video game(s).

The server interface 212 may retrieve one or more questions and/or answers and/or answer options from remote computing devices, such as the server 104 in FIG. 1. The server interface 212 may retrieve diagnosis information created by the AI engine 106 in FIG. 1.

The profile and/or setting data 214 may comprise information such as target lessons associated with a particular user, lessons completed associated with a particular user, approved third-party application(s) associated with a particular user, test results associated with a particular user, diagnoses associated with a particular user, pause and/or question times and/or patterns associated with a particular user, etc.

The administrator 216 may enable and/or disable execution of applications, such as the third-party application(s) 202 in accordance with information in the profile and/or setting data 214. The administrator 216 may cause the user device 200 to display a question received via the server interface 212.

### Processes for Implicit Disability Diagnosis

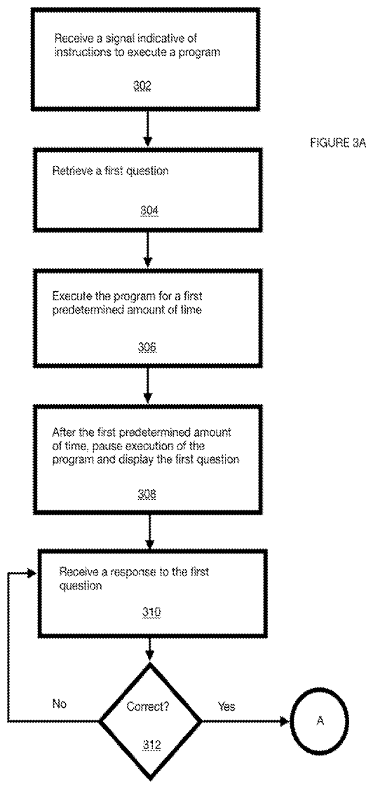

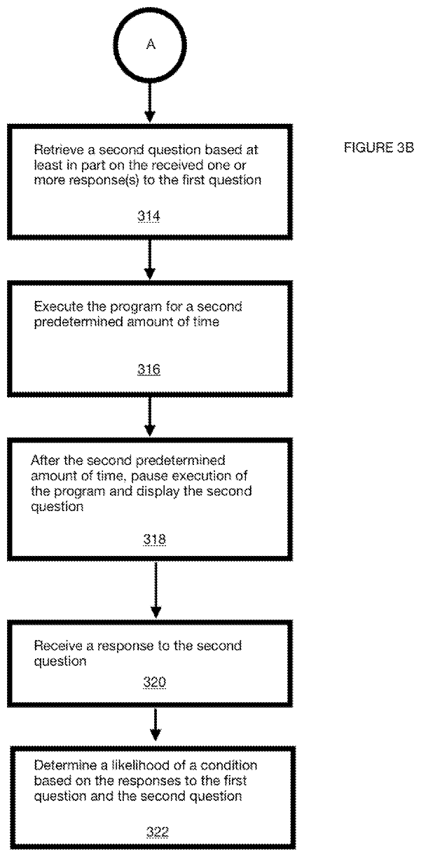

FIGS. 3A and 3B illustrate a flowchart for a method for implicit disability diagnosis in accordance with an exemplary embodiment of the present invention.

At step 302, a signal indicative of instructions to execute a program may be received. For example, the user device 100 in FIG. 1 may receive a signal indicative of instructions to execute a program. The program may be a third-party application. The program may be selected from a preapproved plurality of third-party applications. Execution of the program may cause execution of a video game. Execution of the program may cause display of audio and/or video content.

At step 304, a first question may be retrieved. For example, the user device 100 in FIG. 1 may retrieve a first question from the server 104 in FIG. 1. The first question may test knowledge of a subject.

At step 306, the program may execute for a first predetermined amount of time. For example, the user device 100 in FIG. 1 may execute for a first predetermined amount of time. The first predetermined amount of time may be any appropriate amount of time. As an example, the first predetermined amount of time may be two minutes.

At step 308, after the first predetermined amount of time, execution of the program may be paused and the first question may be displayed. For example, after the first predetermined amount of time, the user device 100 in FIG. 1 may pause execution of the program and may display the first question.

At step 310, a response to the first question may be received. For example, the user device 100 in FIG. 1 may receive a response to the first question.

At step 312, a determination may be made of if the received response to the first question is correct. For example, the user device 100 in FIG. 1 may make a determination of if the received response to the first question is correct. If the received response is determined to be incorrect, then the method may move back to step 310. If the received response is determined to be incorrect, then additional instruction on the subject may be given. For example, a video on the subject may automatically execute in response to an incorrect answer. If the received response is determined to be correct, then the method may move to step 314. Step 312 may be an optional step. In some embodiments, a method may move from step 310 directly to step 314.

At step 314, a second question may be retrieved based at least in part on the received one or more response(s) to the first question. For example, the user device 100 in FIG. 1 may retrieve a second question based at least in part on the received one or more response(s) to the first question. The second question may test knowledge of the subject. The second question may be retrieved based on a number of wrong responses to the first question. The second question may be retrieved based selection of one or more particular wrong responses to the first question. The second question may be retrieved based on a response time associated with a correct response to the first question. Differences in the first question and the second question may be aimed at exposing a condition. The first question may comprise one or more different colors than the second question. The first question may comprise different wording than the second question. The first question and/or the second question may be designed to test a user's patience. The first question and/or the second question may be designed to expose tendencies towards seizures. Exposing a condition may comprise exposing color blindness. Exposing a condition may comprise exposing a learning disability. Exposing a condition may comprise exposing dyslexia. Exposing a condition may comprise exposing attention deficit disorder. Exposing a condition may comprise exposing attention deficit hyperactivity disorder. Exposing a condition may comprise exposing epilepsy.

At step 316, the program may execute for a second predetermined amount of time. The first predetermined amount of time may be the same as the second predetermined amount of time. The first predetermined amount of time may be the different than the second predetermined amount of time. The first predetermined amount of time may be less than the second predetermined amount of time. The first predetermined amount of time may be more than the second predetermined amount of time. The second predetermined amount of time may be based on a number of wrong responses to the first question. The second predetermined amount of time may be based selection of one or more particular wrong responses to the first question. The second predetermined amount of time may be based on a response time associated with a correct response to the first question.

At step 318, after the second predetermined amount of time, execution of the program may be paused and the second question may be displayed. For example, after the second predetermined amount of time, the user device 100 in FIG. 1 may pause execution of the program and may display the second question.

At step 320, a response to the second question may be received. For example, the user device 100 in FIG. 1 may receive a response to the second question.

At step 322, a likelihood of a condition may be determined based on the responses to the first question and the second question. For example, the user device 100 in FIG. 1 may determine a likelihood of a condition based on the responses to the first question and the second question. The condition may comprise one or more of color blindness, a learning disability, dyslexia, attention deficit disorder, attention deficit hyperactivity disorder, and epilepsy. The condition may comprise any condition in which a sample of people with the condition is likely to answer the first question in a first manner and the second question in a second manner.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

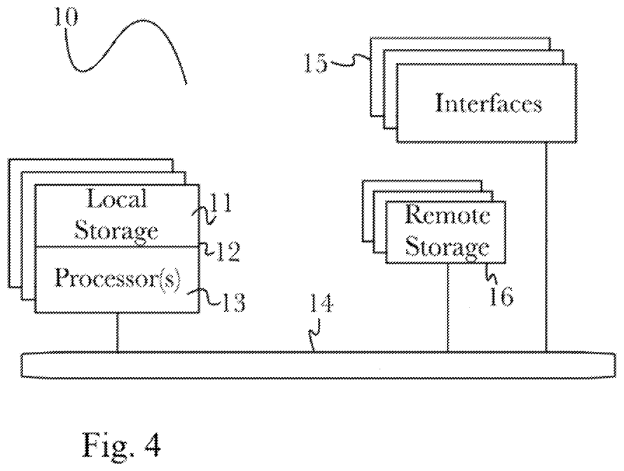

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

The computing device 10 may be and/or comprise the user device 100 in FIG. 1.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

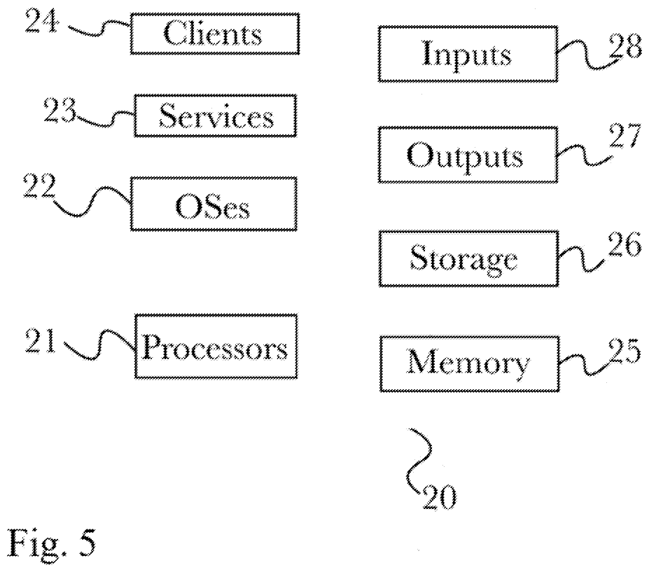

In some embodiments, systems may be implemented on a standalone computing system. Referring now to FIG. 5 above, there is shown a block diagram depicting a typical exemplary architecture of one or more embodiments or components thereof on a standalone computing system. Computing device 20 includes processors 21 that may run software that carry out one or more functions or applications of embodiments, such as for example a client application 24. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications 24. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 5). Examples of storage devices 26 include flash memory, magnetic hard drive, CD-ROM, and/or the like.

The system 20 may be and/or comprise the user device 100 in FIG. 1.

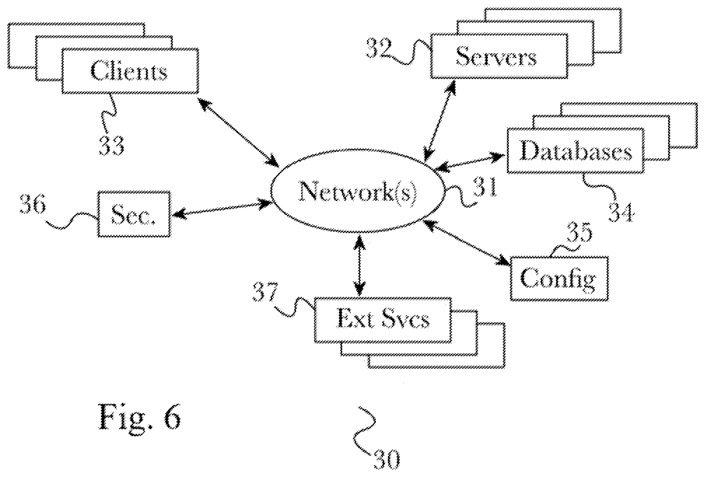

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications 24 are implemented on a smartphone or other electronic device, client applications 24 may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

The one or more of the server(s) 32 may be and/or comprise the user device 100 in FIG. 1.

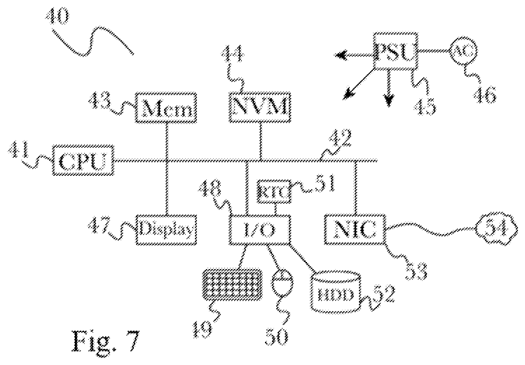

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

The computer system 40 may be and/or comprise the user device 100 in FIG. 1.

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for implicit disability diagnosis through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computer-implemented method for implicitly diagnosing disability, the computer-implemented method comprising:
receiving a first signal;
receiving a responsive first input;
executing a first program for a predetermined amount of time;
display a first question;
determine if a second response is received;
display a second questions and receive a second response; and
compute a disability diagnosis.
